(19) United States
(12) Patent Application Publication
(10) Pub. No.: US 2022/0033803 A1
(43) Pub. Date: Feb. 3, 2022

(54) METHOD OF EXTRACTING MIRNA AND METHOD OF ANALYZING MIRNA

(71) Applicant: NATIONAL UNIVERSITY CORPORATION TOKAI NATIONAL HIGHER EDUCATION AND RESEARCH SYSTEM, Nagoya-shi (JP)

(72) Inventors: Takao YASUI, Nagoya-shi (JP); Yoshinobu BABA, Nagoya-shi (JP)

(21) Appl. No.: 17/289,611

(22) PCT Filed: Oct. 30, 2019
(86) PCT No.: PCT/JP2019/042499

§ 371 Date: Sep. 24, 2021

(30) Foreign Application Priority Data
Oct. 30, 2018 (JP) 2018-203527

(51) Int. Cl. C12N 15/10; C12Q 1/6876

(52) U.S. Cl. CPC C12N 15/1006; C12Q 2600/178; C12Q 1/6876

(57) ABSTRACT

The present invention provides a novel miRNA extraction method and a method for analyzing miRNA extracted by using said miRNA extraction method. According to the present invention, provided is, for example, a method for extracting miRNA from extracellular vesicles in a sample solution, by using a device capable of capturing extracellular vesicles, the miRNA extraction method comprising: an extracellular vesicle capturing step for capturing extracellular vesicles in a sample solution onto a device by bringing the sample solution and the device in contact with each other; and a miRNA extraction step for homogenizing the extracellular vesicles by bringing the device having captured the extracellular vesicles in contact with a homogenization liquid for extracellular vesicles to extract miRNA from the extracellular vesicle into the homogenization liquid.

### TECHNICAL FIELD

The disclosure in this application relates to methods of extracting miRNA and methods of analyzing miRNA. In particular, it relates to miRNA extraction methods for extracting miRNA from EVs in sample solution using a device capable of capturing extracellular vesicles (Extracellular Vesicles, exosomes; hereinafter, sometimes referred to as “EVs”), and methods for analyzing miRNA contained in an extraction solution obtained by a miRNA extraction method.

### BACKGROUND ART

EVs are membrane endoplasmic reticula of about 40-1000 nm in size secreted by cells in vivo and are present in body fluids such as blood, urine, saliva, and semen. Membrane proteins, adhesion molecules, enzymes, and the like derived from secretory cells are present on the surfaces, and nucleic acids such as mRNA and miRNA are contained inside. Therefore, they propagate to other cells and are taken up, thus affecting the recipient cells.

In recent years, it has become clear that EVs induce cancer metastasis as one of their functions in vivo, and this has attracted attention. Cancer metastasis refers to the propagation of cancer cells from the site of cancer to other organs via blood vessels and lymph and the growth, and the high mortality from cancer is also attributable to this metastasis. Regarding the development of this cancer metastasis, researches on EVs and cancer metastasis have been reported, including EVs from cancer cells of the cancer primary lesion propagating through blood vessels to other organs, forming a cancer metastatic niche, and EVs derived from cancer cells inducing abnormal proliferation of normal cells and developing into cancer tumorigenesis (see Non-Patent Literature 1).

It is also known that miRNA contained in EVs are used as a biomarker for diseases [Non-Patent Literatures 2 and 3].

### CITATION LIST

### Non-Patent Literature

Non-Patent Literature 1: Sonia A. Melo, et al., “Cancer Exosomes Perform Cell-Independent MicroRNA Biogenesis and Promote T umorigenesis”, Cancer Cell 26, 707-721, Nov. 10, 2014 http://dx.doi.org/10.1016/j.ccell.2014.09.005
Non-Patent Literature 2: Amanda Michael, et al., “Exosomes from Human Saliva as a Source of microRNA Biomarkers”, Oral Dis. 2010 January; 16(1):34-38. doi:10 0.1111/j.1601-0825.2009.01604.x.
Non-Patent Literature 3: Kazuya Iwai, et al., “Isolation of human salivary extracellular vesicles by iodixanol density gradient ultracentrifugation and their characterizations”, Journal of Extracellular Vesicles 2016, 5:30829-http://dx.doi.org/10.3402/jev.v5.30829

### SUMMARY OF INVENTION

### Technical Problem

As described in Non-Patent Literatures 2 and 3 described above, it is known to use miRNA contained in EVs in a sample (saliva in Non-Patent Literatures 2 and 3) as biomarker for diseases. By the way, it is described in Non-Patent Literatures 2 and 3 that EVs are collected from the sample solution by ultracentrifugation of the sample solution. However, separation by ultracentrifugation requires to collect the fractions containing EVs after the ultracentrifugation.

Therefore, there is a problem that an ultracentrifugation step is essential, and the work procedure increases. Furthermore, when the amount of the sample solution is small, in order to analyze a trace amount of miRNA contained in the sample solution, it is necessary to reduce the loss when collecting EVs contained in the sample solution. However, in methods of collecting EVs by ultracentrifugation, there is a problem that a part of EVs contained in a sample may be discarded during the operation process of collecting the fraction containing EVs. Further, as a method for separating EVs in a sample solution, an aggregation reagent method using a commercially available kit is also known in addition to the ultracentrifugal method. However, even with respect to the aggregation reagent method, after aggregating EVs in the sample solution, it is necessary to separate the aggregated EVs by centrifugation or the like. Thus, there is a problem that the work procedure increases and a loss occurs during the operation of separation of EVs. Therefore, there is a need for a method of collecting EVs from a sample solution in a simple and efficient manner.

The disclosure of the present application has been made to solve the above-mentioned problems, and as a result of intensive studies, it has been newly discovered that [1] the EVs can be captured by a device by contacting the sample solution with a device capable of capturing EVs, [2] by contacting the device having captured EVs directly with the EV disruption solution, [3] miRNA can be directly extracted from the EVs captured by the device, without requiring a step of separating the EVs captured by the device.

In other words, it is an object of the disclosure of the present application to provide a new method for extracting miRNA and an analysis method for analyzing miRNA extracted by the method for extracting miRNA.

### Solution to Problem

The disclosure of the present application relates to methods of extracting miRNA and methods of analyzing miRNA, shown below.

(1) A method for extracting miRNA from extracellular vesicles in a sample solution using a device capable of capturing extracellular vesicles, the method comprises:

an extracellular vesicle capture step of bringing the sample solution into contact with the device, to capture an extracellular vesicle in the device; and

a miRNA extraction step of bringing the device that captured the extracellular vesicle with the disruption solution of extracellular vesicles, to disrupt the extracellular vesicle and extract miRNA from the extracellular vesicle into the disruption solution.

(2) The method of extracting miRNA, according to (1) above, comprising:

a device cleaning step of cleaning the device that captured the extracellular vesicle, between the extracellular vesicle capture step and the miRNA extraction step.

(3) The method of extracting miRNA, according to (1) or (2) above, wherein the device is formed of a material which is resistant to a disruption solution.

(4) The method of extracting miRNA, according to any one of (1) to (3) above, wherein the device comprises a nonwoven fabric composed of cellulose fibers.

(5) The method of extracting miRNA, according to (4) above, wherein the cellulose fiber is a cellulose nanofiber.

(6) The method of extracting miRNA, according to (3) above, wherein the device comprises at least one selected from:

a nanowire,

a structure made with cellulose fibers, and

a structure made with cellulose nanofibers.

(7) The method of extracting miRNA, according to (6) above, wherein the device is a structure made with cellulose nanofibers.

(8) The method of extracting miRNA, according to any one of (1) to (7) above, wherein the sample solution is a non-invasive biological sample solution.

(9) The method of extracting miRNA, according to (8) above, wherein the sample solution is saliva.

(10) A method of analyzing miRNA contained in an extracellular vesicle in a sample solution, comprising an analysis step of analyzing miRNA contained in the disruption solution extracted by the miRNA extraction method of miRNA according to any one of (1) to (9) above.

### Advantageous Effects of Invention

By the miRNA extraction methods disclosed in the present application, the step of separating EVs in the sample solution by ultracentrifugation or the like is not required, and miRNA can be extracted directly from EVs captured in a device. In addition, by analyzing miRNA extracted by the miRNA extracting methods, a trace amount of miRNA can also be analyzed.

### BRIEF DESCRIPTION OF DRAWINGS

FIG. 1 shows a flowchart of an extraction method according to the first embodiment.

FIGS. 2A to 2D show an example of device 1 according to the third embodiment.

FIG. 3 shows a manufacturing process of device 1a having nanowires 3 formed on the first surface of the substrate 2 as an example of device 1 according to the third embodiment.

FIGS. 4A to 4C show various aspects of cover member 4. FIG. 4D shows substrate 2 having nanowires formed on the first surface.

FIG. 5 explains an example of a manufacturing process of device 1b according to the fourth embodiment.

FIGS. 6A to 6E are photographs in substitution for drawing showing manufactured devices 1 to 5, respectively.

FIGS. 7A and 7B are photographs in substitution for drawing showing (a) the centrifuge tube after the device was removed from the centrifuge tube after miRNA were extracted, and (b) the device removed from the centrifuge tube.

FIGS. 8A and 8B are photographs in substitution for drawing showing (a) a photograph of a centrifuge tube immediately after completion of miRNA extraction step; (b) a photograph of a centrifuge tube after removal of the device from the centrifuge tube after miRNA extraction; and (c) a photograph of the device removed from the centrifuge tube.

FIG. 9 shows a graph showing the type of miRNA contained in miRNA extract solution extracted using devices 1 to 4.

### DESCRIPTION OF EMBODIMENTS

Hereinafter, referring to the drawings, miRNA extraction methods (hereinafter, sometimes simply referred to as “extraction methods”) and miRNA analysis methods (hereinafter, sometimes simply referred to as an “analysis methods”) will be described in detail. Note that, in the present specification, members having the same kind of functions are denoted by the same or similar reference numerals. Repeated descriptions of the same or similar numbered members may be omitted.

### First Embodiment of Extraction Method

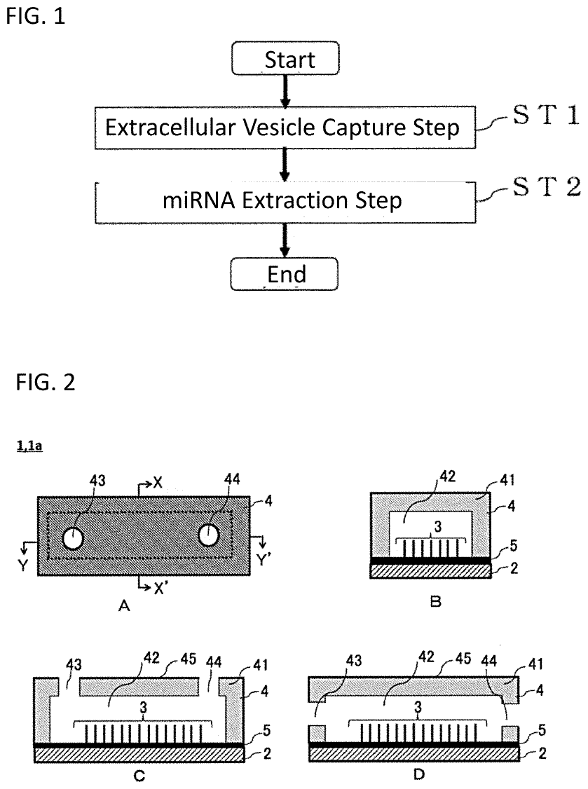

Referring to FIG. 1, the first embodiment of the extraction method will be described. FIG. 1 shows a flowchart of the extraction method according to the first embodiment. The first embodiment of the extraction method includes an extracellular vesicle (EVs) capture step (ST1), a miRNA extraction step (ST2)

In the extracellular vesicle (EVs) capture step (ST1), by contacting the sample solution with a device capable of capturing EVs, EVs in the sample solution are captured in the device. In the miRNA extraction step (ST2), by contacting the device that captured EVs with the EVs disruption solution, EVs are disrupted and miRNA are extracted from the EVs into the disruption solution.

The sample solution is not particularly limited as long as it contains EVs and may be a biological sample solution such as blood, lymph, bone marrow fluid, semen, breast milk, amniotic fluid, urine, saliva, nasal mucus, sweat, tears, bile fluid, cerebrospinal fluid, or the like. Further, examples of the sample solution other than biological sample solutions include a cell culture supernatant, a sample solution for an experiment in which EVs are added to a medium or a buffer solution, and the like. When a biological sample solution is used as a sample solution, a non-invasive sample solution such as urine, saliva, nasal mucus, sweat, or tear is preferred in consideration of reduction in patient burden.

Note that, as shown in the examples described later, miRNA extracted in the first embodiment of the extraction method disclosed in the present application was analyzed, and many types of miRNA could be analyzed. In other words, even a trace amount of miRNA that could not be analyzed by conventional methods could be analyzed. Therefore, if a sample solution of the same type is used, the extraction method can be performed in a small amount. In addition, in order to fractionate and collect EVs by ultracentrifugation, a sample solution of about several milliliters is required. However, there are also biological sample solutions, such as saliva and tears, for example, in which it becomes a great burden for the patient to collect a quantity of several milliliters. In the first embodiment of the extraction method, since miRNA can be extracted even if the amount of the sample solution is small as compared with the conventional ultracentrifugation methods, it is particularly useful for extracting miRNA contained in the EVs in saliva.

There is no particular limitation on the disruption solution of EVs as long as EVs can be disrupted, and for example, a commercially available cell lysis buffer (Cell Lysis Buffer) may be used. Examples of the cell lysis buffer include cell lysis buffer M (manufactured by Fujifilm Wako Pure Chemical Industries, Ltd., 038-21141), RIPA Buffer (manufactured by Fujifilm Wako Pure Chemical Industries, Ltd., 182-02451), and the like. Note that, the time for immersing the device in the disruption solution is not particularly limited as long as miRNA can be taken out by disrupting the EVs. The device will be described later.

### Second Embodiment of Extraction Method

The second embodiment of the extraction method differs from the first embodiment of the extraction method in that between the extracellular vesicle (EVs) capture step (ST1) and the miRNA extraction step (ST2) shown in FIG. 1, a device cleaning step of cleaning the device that captured EVs is included, and the other points are similar to those of the first embodiment of the extraction method. The biological sample solution extracted from the living body, for example, saliva, sweat, nasal mucus, and the like, contains RNase, which is an enzyme for decomposing RNAs of foreign substances such as viruses, in order to protect the living body from viruses and the like entering from the outside. Therefore, when RNase is extracted from a biological sample solution containing miRNA such as saliva, perspiration, and nasal water, there is a risk that RNase is adsorbed on the device during the extracellular vesicle capture step. Then, there is a risk that RNase decomposes miRNA extracted from the EVs when miRNA extraction step is performed on the device to which RNase is adsorbed.

Therefore, in the device cleaning step, RNase is removed from the device by cleaning the device that captured EVs. In the device cleaning step, the device that captured EVs may be immersed in a cleaning solution for a predetermined time and washed. The cleaning time is not particularly limited, but if it is too short, there is no cleaning effect, and if it is too long, there arises a problem that the captured EVs are peeled off. For example, the device may be immersed in a cleaning solution for about 1 to 2000 seconds. Examples of the cleaning solution include pure water, PBS, NaCl, physiological saline, and various buffers such as PBS. Note that, when pure water is used as a cleaning solution, if the cleaning is performed for a long time, there is a risk that EVs captured may burst in the relationship of osmotic pressure. Therefore, when pure water is used as the cleaning solution, it is desirable to set the cleaning time to be shorter as compared with a buffer or the like.

### Embodiments of miRNA Analysis Methods

Embodiments of the analysis methods of miRNA include an analysis step of analyzing miRNA in the disruption solution extracted according to the first or the second embodiment of the extraction method. For analysis of miRNA, known miRNA analysis methods may be used. For example, methods may be used as follows: (1) total RNA including miRNA are extracted using miRneasy Mini Kit(QIAGEN), exhaustive analysis is performed from about 2500 types of miRNA using a 3D-Gene (registered trademark) miRNA chip, chip images are digitized, expression ratios are calculated, variable genes are analyzed, and cluster analysis is performed, (2) miRNeasy Serum/Plasma kit (Qiagen) is used to isolate total miRNA in a disruption solution, miScript II RT Kit (Qiagen) is used to synthesize cDNA, and quantitative real-time PCR is performed.

Hereinafter, a device which can be used in a method of extracting miRNA disclosed in this application will be described. The device is not particularly limited as long as it can capture EVs, but the device can include a “nanostructure body” in order to improve the capture efficiency of EVs and the like. In this specification, the term “nanostructure body” means a structure body capable of adsorbing EVs by interaction, and enhancing the adsorption efficiency of EVs by increasing the specific surface area as compared with the minimum area of materials of the same kind and in the same amount. The nanostructure body can be manufactured, for example, by using a material having fine pores (nanopores), or by aggregating (clustering) fine fibers (wires), or the like. The shape of the nanostructure body is not particularly limited, and may be any of, for example, a film shape; a thread (string) shape; a cylindrical shape, a prismatic shape, a three-dimensional shape such as an irregular shape, or the like. Embodiments of the film-like and nanowire-based devices will be described below, but the following device embodiments are merely illustrative, and the devices are not limited to the embodiments illustrated below.

### First Embodiment of Device

The first embodiment of the device uses a film manufactured using cellulose nanofibers as the nanostructure body. To obtain cellulose nanofibers, wood fibers (cellulose fibers) are first removed from wood chips and pulped. This cellulose fiber is composed of myriad cellulose nanofibers in bundles. Next, in the presence of a TEMPO catalyst, the cellulose fibers are collided with each other at a high pressure in a solvent to dissolve the bundled cellulose fibers, thereby obtaining cellulose nanofibers. Note that the method for manufacturing the cellulose nanofibers described above is merely exemplary, and other methods may be used. The device according to the first embodiment can be manufactured by subjecting a solvent containing the obtained cellulose nanofibers to suction filtration, whereby the cellulose nanofibers are aggregated and formed into a film by surface tension. Examples of the solvent for dispersing the cellulose nanofibers include water and the like.

Note that, in the device according to the first embodiment, the cellulose nanofibers of the manufactured film may have gaps (nanopores). By adjusting the size of the nanopores, it is possible to improve the capture efficiency of EVs. The nanopore size can be, for example, about 1 nm to 200 nm, about 1 nm to 100 nm. The average size of the nanopores can be measured by mercury intrusion. Nanopores can be formed by applying a liquid having a low surface tension, such as tertiary butyl alcohol, ethanol, or isopropanol, to a wet state cellulose nanofiber which have been subjected to suction filtration and aggregated, and subsequently sucking, and replacing and drying the solvent contained in a mass of aggregated cellulose nanofibers with such a solvent having a low surface tension. The size of the nanopores can be adjusted by varying the solvent to be added. The formation and size adjustment of the nanopores described above are merely examples, and the formation and size adjustment of the nanopores may be performed by other methods. For example, by changing the high pressure treatment conditions for dissolving the cellulose fibers or by changing the cellulose raw material such as the type, bacteria, and ascidia of the pulp, the width of the cellulose nanofibers and the size of the nanopores may be adjusted. In an aspect, the manufactured film can be a nonwoven fabric. When dispersing nanopores, more EVs are captured, and many types of miRNA can be analyzed.

### Second Embodiment of Device

The second embodiment of the device differs from the first embodiment in that a film manufactured using cellulose fibers (pulp) are used as the nanostructure body instead of cellulose nanofibers. The device according to the second embodiment may be manufactured by the same procedure as in the first embodiment of the device, except that the cellulose fibers (pulp) are dispersed in a solvent instead of the cellulose nanofibers. The gap between the cellulose fibers and the gap between the cellulose nanofibers present on the cellulose fiber surface can also be manufactured and the size can be adjusted in the same manner as in the first embodiment. Since the width of the cellulose nanofibers is about 3 nm to 100 nm, nanopores having a size of about 1 nm to 200 nm are formed. On the other hand, the width of the cellulose fiber is about 20 μm to 40 μm. Thus, unlike the first embodiment, the size of the gap is multi-scaled, on the order of nm to μm, on the order of about 1 nm to 200 nm, and on the order of about 1 μm to 100 μm.

In the device according to the first and second embodiments, the manufactured film can be cut into an appropriate size and used as it is. Alternatively, a device which has been cut may be attached into a centrifuge tube or the like used in the miRNA extraction step described later, it can be sticked to a mask to capture EVs in the cough, and it can be sticked to a towel or the like to capture EVs in the sweat. Also, although the first and second embodiments of the device are film-like, they may be of other shapes. For example, in the case of forming a thread (string), a mold in which a groove is formed in the form of a thread (string) (suction filtration filter) may be used when suction filtration is performed. Further, a solvent in which cellulose (nano) fibers are dispersed may be injected into a coagulation bath such as acetone and spun. In the case of forming a predetermined three-dimensional shape, suction filtration may be performed using a mold (suction filtration filter) in which a predetermined shape is formed. In addition, when forming an irregular three dimensional shape is formed, first, a solvent in which cellulose (nano) fibers are dispersed is charged into only a part of a suction filtration filter, and a mass of cellulose (nano) fibers aggregated is manufactured by suction filtration, and the manufacture of the mass of aggregated cellulose (nano) fibers is repeated, and thereby an irregular shape of three dimensional nanostructure body can be manufactured. Also, a solvent in which cellulose (nano) fibers are dispersed can be placed in a container having a desired shape, and a freeze-drying treatment can be performed, to manufacture a nanostructure having a three-dimensional shape. Further, the device may be manufactured of only cellulose (nano) fibers, or a filler or the like may be added as long as it does not impair the purpose of the present disclosure. Examples thereof include the addition of a filler such as polyamidoamine epichlorohydrin as a wet paper force enhancer, the addition of nanowires (for nanowires, see the third embodiment described later) alone, and the like.

### Third Embodiment of Device

The third embodiment of the device uses nanowires as a device. FIGS. 2A to 2D illustrate an example of devices 1 according to the third embodiment. FIG. 2A shows a top view of device 1a, FIG. 2B shows a X-X′ cross-sectional view, and FIG. 2C shows a Y-Y′ cross-sectional view. Further, FIG. 2D shows a cross-sectional view of a modification of the embodiment shown in FIG. 2C. The device 1a includes at least a substrate 2, a nanowire 3, and a cover member 4, and the device 1a shown in FIG. 2B to FIG. 2D (hereinafter, the descriptions common to FIG. 2 may simply be described as “FIG. 2”. The same applies to the following paragraphs.) includes a catalyst layer 5 for forming the nanowires 3. The device 1a has the catalyst layer 5 formed on the substrate 2 for forming the nanowires 3, the nanowires 3 are formed on the catalyst layer 5. In this specification, the “first surface” means the outermost surface of the surface of the side on which the nanowires 3 of the substrate 2 are formed. Therefore, as described later, when the “first surface” of the substrate 2 and the “second surface” of the cover member are described as being in liquid-tight contact with each other, the member of the “first surface” becomes the substrate 2, the catalyst layer 5, or the coating layer, according to the manufacturing method. Furthermore, in some cases the nanowires are grown on the “first surface” to be in close contact with the “second surface” of the cover member, in which case the flat portion at the base of the nanowires becomes the “first surface”. Also, as used herein, the term “tip” of a nanowire refers to the end of the nanowire away from the first surface of the substrate 2, of both ends of the nanowire, and the end of the nanowire on the first surface side of the substrate 2 is referred to herein as “end.”

The cover member 4 includes a cover member base material 41 and a flow path 42 formed in the cover member base material 41. In this specification, the “second surface” means a surface of the cover member base material 41 on the side where the flow path 42 is formed (in the case where the opening portion of the flow path 42 is a virtual plane, a surface following the virtual plane). In the example shown in FIG. 2B, the surface of the cover-member base material 41 in contact with the catalytic layer 5 corresponds to the second surface. In the embodiment shown in FIG. 2C, the cover member 4 includes a sample introduction hole 43 and a sample collection hole 44. As shown in FIG. 2C, the sample introduction hole 43 and the sample collection hole 44 are formed in the cover member base material 41 so as to penetrate the flow path 42 and the surface 45 opposed to the second surface. Moreover, the example shown in FIG. 2C shows an example of introducing and collecting the sample solution from above of the device 1a, but the positions of the sample introduction hole 43 and the sample collection hole 44 are not particularly limited as long they can collect the sample solution which was input and passed the region with formed nanowires 3, can be collected after passing there. For example, as shown in FIG. 2D, the sample introduction hole 43 and the sample collection hole 44 may be formed in the side wall of the flow path 42.

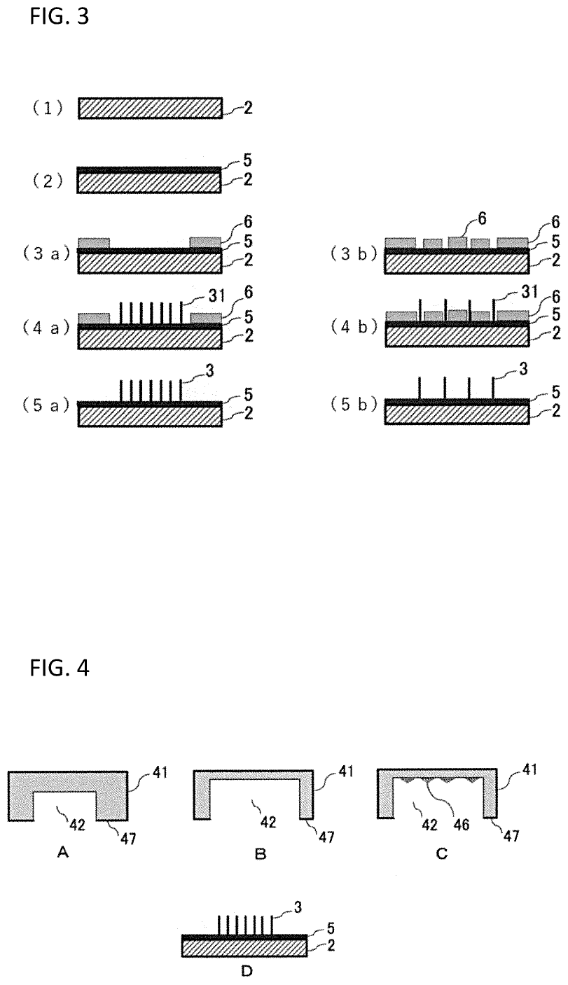

The device 1a according to the third embodiment can be manufactured using a photolithography technique. FIG. 3 shows an example of the device 1 according to the third embodiment, for explaining an example of a manufacturing process of the device 1a having the nanowires 3 formed on the first surface of the substrate 2. FIG. 3 illustrates a cross-sectional view of X-X′ in FIG. 2A.

(1) Prepare a substrate 2.

(2) Form the catalyst layer 5 on the substrate 2, by sputtering particles for manufacturing nanowire 3 by ECR (Electron Cyclotron Resonance) sputtering, or the catalyst ECR sputtering, depositing by EB (Electron Beam) deposition, PLD (Pulsed Laser Deposition), ALD (Atomic Layer Deposition). In this specification, the term “catalyst layer” means “particle” or “layer” of “catalyst” for manufacturing nanowires.

(3a, 3b) Apply resist 6 for photolithography, and pattern by photolithography the location where the nanowires 3 are to be grown. The patterning of the photolithography may be formed in a pattern in which the nanowires 3 are to be grown. For example, if the nanowires 3 are to be grown at random, it may be patterned so that the catalyst layer 5 of the region forming the nanowires 3 on the substrate 2 is all exposed (see 3a). Further, when the nanowires 3 are to be grown at predetermined intervals, a patterning or a drawing by photolighography may be done so as to expose the catalyst layer 5 in the shape of dots at predetermined intervals (see 3b). After patterning or drawing by photolithography, the resist 6 of the patterned or drawn portion is developed and removed.

(4a, 4b) the resist is removed to grow the nanowires 3 from where the catalyst layer 5 is exposed.

(5a, 5b) by removing the remaining resist, it is possible to manufacture a substrate 2 having nanowires 3 formed on the catalyst layer 5 formed on the first surface.

FIGS. 4A to 4C show various aspects of the cover member 4. The cover member 4 can be easily manufactured by cutting the second surface 47 of the cover member base material 41 or pressing a convex mold against the material of the cover member base material 41. When the cover member 4 is manufactured by pressing a convex mold, the sample introduction hole 43 and the sample collection hole 44 may be formed by using a biopsy trepan, an ultrasonic drill, or the like after transfer. By changing the cutting area and the shape of the mold of the cover member 4, for example, as shown in FIG. 4A and FIG. 4B, the cross-sectional area of the flow path 42 can easily be changed. As shown in FIG. 4C, a non-planar area 46 may be formed for generating turbulence in the sample solution passing through on any surface of the flow path 42. The nonplanar area 46 is not particularly limited as long as it can generate turbulence in the sample solution passing therethrough, and for example, a convex portion or the like may be formed. The cover members 4 can be prepared, in a plurality of different types in the cross-sectional area and the shape of the flow path 42.

Then, the substrate 2 (FIG. 4D) having nanowires 3 formed on the first surface prepared by the process shown in FIG. 3 can be covered by the cover member 4 having a flow path 42 of a desired cross-sectional area and shape, to manufacture a device 1a.

The substrate 2 is not particularly limited as long as the catalyst layer 5 can be laminated. Examples include silicon, quartz glass, Pyrex (registered trademark) glass, and the like.

Regarding the catalyst layer 5, the particles for preparing the nanowires 3 may be, for example, ZnO. Examples of the catalyst for manufacturing the nanowires 3 include gold, platinum, aluminum, copper, iron, cobalt, silver, tin, indium, zinc, gallium, chromium, oxides thereof, and the like.

The resist 6 for photolithography is not particularly limited as long as it is commonly used in the semiconductor field, such as OFPR8600LB, SU-8 and the like. Further, as the removing liquid of the resist 6, there is no particular limitation as long as it is a removing liquid common in the semiconductor field such as dimethylformamide and acetone.

The nanowires 3 may be grown from the catalyst layer 5 by a known method. For example, when using ZnO fine particles as the catalyst layer 5 they can be manufactured using a hydrothermal synthesis method. Specifically, by immersing the heated substrate 2 in a precursor solution in which zinc nitrate hexahydrate (Zn(NO₃)₂.6H₂O) and hexamethylenetetramine (C₆H₁₂N₄) are dissolved in deionized water, ZnO nanowires 3 can be grown from a portion where ZnO particles (catalyst layer 5) are exposed.

When a catalyst is used as the catalyst layer 5, the nanowires 3 can be manufactured in the next step.

(a) Using materials such as SiO₂, Li₂O, MgO, Al₂O₃, CaO, TiO₂, Mn₂O₃, Fe₂O₃, CoO, NiO, CuO, ZnO, Ga₂O₃, SrO, In₂O₃, SnO₂, Sm₂O₃, EuO, etc., the core nanowires are formed by a physical vapor deposition method such as pulsed laser deposition, VLS (Vapor-Liquid-Solid) method.

(b) Using SiO₂, TiO₂ or the like, sputtering, EB (Electron Beam) deposition, PVD (Physical Vapor Deposition), by a common deposition method such as ALD (Atomic Layer Deposition), to form a coating layer around the core nanowires. Note that the coating layer of (b) above is not essential and may be implemented as necessary.

The diameter of the nanowires 3 may be appropriately adjusted according to the purpose. When forming using ZnO fine particles, the diameter of the nanowire 3 may be changed by the size of the ZnO fine particles. When forming a coating layer on the manufactured nanowires 3, the diameter can be appropriately adjusted by changing the deposition time when forming the coating layer.

As a material for manufacturing the cover member 4, there is no particular limitation as long as it can be cut or transfer the mold. Examples include: thermoplastic resins such as polyethylene, polypropylene, polyvinylchloride, polyvinylidene chloride, polystyrene, polyvinyl acetate, polytetrafluoroethylene, ABS (acrylonitrile butadiene styrene) resins, AS (acrylonitrile styrene) resins, acrylic resins (PMMA), and the like; thermosetting resins such as phenolic resins, epoxy resins, melamine resins, urea resins, unsaturated polyester resins, alkyd resins, polyurethanes, thermosetting polyimides, and silicone rubbers, and the like.

The examples shown in FIGS. 2 to 4 are merely exemplary of the device 1, there is no particular limitation as long as the nanowires are formed on the substrate 2. For example, nanowires may be formed in the flow paths formed on the substrate 2 by the procedure described in WO 2015/137427.

### Fourth Embodiment of Device

The device 1b according to the fourth embodiment is different from the device 1a according to the third embodiment in that the end portion of the nanowire 3 is embedded in the first surface of the substrate 2a and in that the material for manufacturing the substrate 2a is different from the device 1a according to the third embodiment, and is otherwise the same as the device 1a according to the third embodiment.

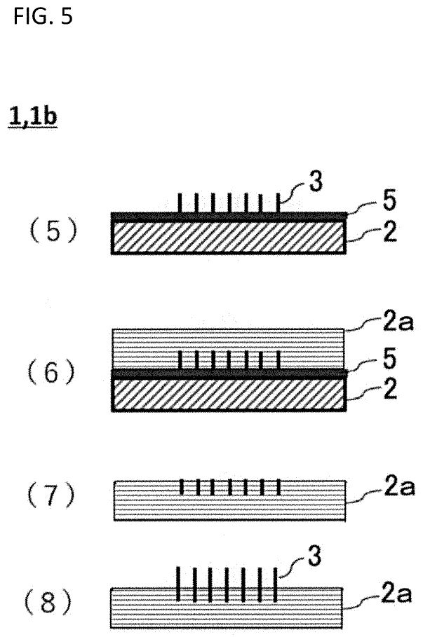

FIG. 5 is a drawing for explaining an example of a manufacturing process of the device 1b according to the fourth embodiment;

(5) Prepare a substrate 2 having formed with nanowires 3 formed on the first surface, which was manufactured in the device 1a according to the third embodiment, as a mold.

(6) Apply the material forming the substrate 2a to the mold.

(7) By peeling the substrate 2a from the mold, form substrate 2a having a portion of the nanowires 3 embedded in the first surface.

(8) By further growing the nanowires 3 embedded in the first surface of the substrate 2a, manufacture the substrate 2a having the end portion of the nanowires 3 embedded in the first surface. The nanowires 3 can be grown by the same procedure as in the first embodiment.

Though not shown, the device 1b can be manufactured by covering the substrate 2a with the cover member 4 manufactured in the same procedure as in the third embodiment.

The material for forming the substrate 2a is not particularly limited as long as the nanowires 3 can be embedded, and for example, a material similar to that of the cover member 4 can be used.

When a film-like device shown in the first and second embodiments is used as the device, a sample solution may be dropped to the film or the film may be immersed in the sample solution, in the extracellular vesicle capturing step (ST1). When the devices 1a and 1b in which the nanowires are formed on the substrates of the third and fourth embodiments are used as the devices, the sample solution may be introduced through the sample introduction hole in the extracellular vesicle capture step (ST1).

Then, when a film-like device shown in the first and second embodiments is used as the device, the film may be immersed in the disruption solution in the miRNA extraction step (ST2). When a device in which nanowires are formed on the substrate of the third and fourth embodiments is used as the device, the disruption solution may be introduced through the sample introduction hole and the disruption solution containing the extracted miRNA may be collected in the miRNA extraction step (ST2).

In the devices 1a and 1b according to the third and fourth embodiments, the cover member 4 is formed, but the cover member 4 may not be disposed. In such cases, in the extracellular vesicle capture step (ST1), the sample solution may be dropped to the nanowires or the device may be immersed in such a manner that the nanowires contact the container containing the sample solution. In the miRNA extraction step (ST2), the nanowire part may be immersed in the container containing the disruption solution.

Furthermore, in the devices 1a and 1b according to the third and fourth embodiments, the nanowires 3 are formed on the first surface of the substrate, but the nanowires 3 may be used alone. In such cases, in the extracellular vesicle capture step (ST1), the nanowires may be put into tubes or the like in which the sample solution is put, so that the nanowires and the sample solution are contacted with each other. In addition, in the miRNA extraction step (ST2), after removing the sample solution from the tube, the disruption solution may be introduced into the tube. Even when the nanowires 3 are used alone as a device, miRNA can be directly extracted from EVs captured by the device. When the nanowires 3 are used alone as a device, for example, the nanowires 3 may be collected from the first surface of the substrate.

As shown in the examples described later, the devices shown in each of the above embodiments are capable of capturing EVs in a sample solution. When the EVs are disrupted by the disruption solution and the extracted miRNA are analyzed comprehensively, if the device is broken by the disruption solution, the residue of the disruption may adversely affect the analysis process in the analysis process. Thus, the device may be durable against the disruption solution, e.g., may have durability against the disruption solution for at least 5 minutes, preferably 30 minutes or more. In the above embodiments, films composed of nanowires or cellulose nanofibers are more preferred devices because they are durable against disruption solution.

Note that the above devices are merely illustrative, and are not limited to the devices of the above embodiments as long as they can adsorb EVs (preferably durable against disruption solution). Such devices include porous materials having a large number of pores at the surface. Specific examples thereof include microporous materials such as activated carbon, zeolite and the like, mesoporous materials such as silicon dioxide (mesoporous silica), aluminum oxide and the like, and macroporous materials such as pumice and the like. In addition, other than porous materials, they include a filter made of molten glass or a polymer.

The following examples are provided to explain embodiments disclosed in the present application, but the examples are merely for explanations of the embodiments. It is not intended to limit or restrict the scope of the inventions disclosed in this application.

### EXAMPLES

[Device Fabrication]

<Device 1>

A film-like device having nanopores was manufactured from cellulose nanofibers by the following procedure.

(1) 400 mg of nanocellulose having a width of 15 to 100 nm obtained by treating conifer bleached kraft pulp with a wet pulverization equipment (Star Burst HJP-25005E) manufactured by SUGINO MACHINE Ltd., was introduced into 200 mL of water to obtain a nanocellulose aqueous dispersion.

(2) The above nanocellulose aqueous dispersion was filtered and dehydrated using a filtration device (KG-90, Advantek Toyo Roshi Kaisha, Ltd.) and an aspiration device (Aspirator AS-01, AS ONE Corporation) and through a hydrophilic polytetrafluoroethylene (PTFE) membrane filter (H020A090C, Advantek Toyo Roshi Kaisha, Ltd.).

(3) Subsequently, a solvent replacement step was performed in which 200 mL of tertiary butyl alcohol (^{t}BuOH, 06104-25, Nacalai Tesque Inc.) was dropped onto the dehydrated nanocellulose aggregate and filtered.

(4) The obtained nanocellulose aggregate in a wet state was subjected to a hot press drying treatment (AYSR-5, Shinto Metal Industries, Ltd.) under conditions of 110 □, 1 MPa, and 15 min, and then peeled off from a PTFE membrane filter to obtain a film.

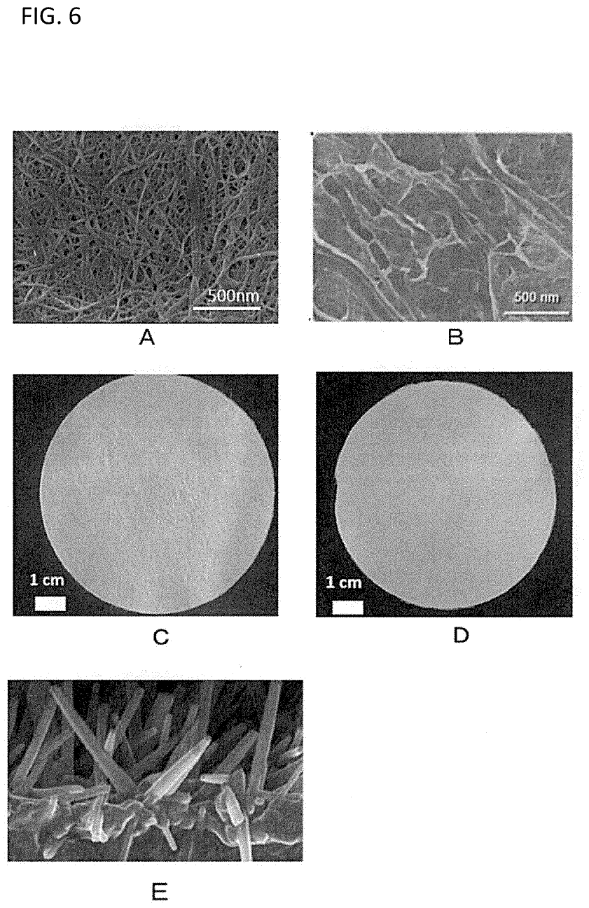

(5) The manufactured film was cut into a square having one side of 1 cm, to manufacture the device 1. FIG. 6A shows an SEM photograph of the manufactured device 1. The nanopore size of the manufactured film was about several nm to 100 nm.

<Device 2>

A film-like device was fabricated from cellulose nanofibers in the same manner as for the device 1, except that the replacement step by ^{t}BuOH of the device 1 was not performed. FIG. 6B shows an SEM photograph of the manufactured device 2. As is apparent from the photograph, the device 2 did not form nanopores between the cellulose nanofibers.

<Device 3>

A film-like device with micro-sized pores was manufactured from pulp (cellulose fiber) by the following procedure.

(1) 400 mg of conifer bleached kraft pulp was introduced into 200 mL of water to obtain a pulp aqueous dispersion.

(2) The above pulp aqueous dispersion was filtered and dehydrated using a filtration device (KG-90, Advantek Toyo Roshi Kaisha, Ltd.) and an aspiration device (Aspirator AS-01, AS ONE Corporation) and through a stainless-steel mesh filter (SUS304, 300 mesh, Clever Inc.).

(3) Subsequently, a solvent replacement step was performed in which 200 mL of tertiary butyl alcohol (^{t}BuOH, 06104-25, Nacalai Tesque Inc.) was dropped onto the dehydrated pulp aggregate and filtered.

(4) The obtained wet pulp aggregate was subjected to a hot press dry treatment (AYSR-5, Shinto Metal Industries, Ltd.) under conditions of 110□, 1 MPa and 15 min, and then peeled off from the stainless mesh filter to obtain a film.

(5) The manufactured film was cut into a square having one side of 1 cm, to manufacture the device 3. FIG. 6C shows a photograph of the manufactured device 3. The pore sizes of the manufactured films were multi-scale of about several nm to 100 nm and about 1 μm to 100 μm.

<Device 4>

A film-like device was fabricated from pulp (cellulose fiber) in the same manner as for the device 3, except that the replacement step by ^{t}BuOH of the device 3 was not performed. FIG. 6D shows a photograph of the manufactured devices 4. The pore size of the manufactured film was about 1 μm to 100 μm.

<Device 5>

A device embedded in a flow channel in which nanowires were formed on a substrate was manufactured by the following procedure. (1) First, a channel patterning of a PDMS embedded nanowire device was done on the Si(100) substrates (Advantech Co, Ltd.). A positive resist (OFPR-8600 LB, Tokyo Ohka Kogyo Co. Ltd.) was spin-coated on the Si substrate surface under conditions of 500 rpm (5 sec) and 3000 rpm (120 sec) by a spin coater (MS-A100, Mikasa Corporation), and then the Si substrate surface was heated on a hot plate at 90□ for 12 min to evaporate the solvent and fix the resist on the substrate. A glass mask was placed on the heated substrate, and the resist was softened by irradiating the substrate with i-line of 600 mJ/cm² by an exposure machine. Finally, the softened resist was removed by immersing the substrate in a developer (NMD-3, Tokyo Ohka Kogyo Co., Ltd.) for about 10 seconds, and the substrate was taken out of the developer and washed with flowing water. Then, the substrate was heated at 90□ for 5 min on a hot plate to complete the patterning.

(2) Next, a Cr layer was made, which becomes a seed layer of the nanowire growth on the substrate surface. With the condition of the sputtering device (EIS-200ERT-YN, Elionics Corporation) for preparing the Cr layer was 1.2×10⁻² Pa for 14 min, a 135 nm-thick Cr layer was deposited. The substrate was immersed in 2-propanol warmed to 70□ on a hot plate for 40 min, and then subjected to an ultrasonic treatment for 2 min with an ultrasonic instrument to roughly remove the resist outside the flow path. Thereafter, the substrate was transferred to 2-propanol at 70□ placed in another container, and after immersion for 10 min, the resist outside the flow path was completely removed by performing an ultrasonic treatment for 1 min. Finally, fine Cr particles on the substrate were removed by rinsing in 2-propanol at 70□ in another container. By these steps, the Cr layer deposition was only in the flow path portion on the substrate. This substrate was heated in an electric furnace at 400□ for 2 h to oxidize the Cr layer and to complete the seed layer manufacture of the nanowire growth.

(3) To 200 mL of ultrapure water, hexamethylenetetramine (HMTA; 085-00335, Wako Pure Chemical Industries, Ltd.) was dissolved so as to become 15 mM, and it was stirred by a stirrer for 7 min. Thereafter, the solution was further dissolved so that zinc nitrate hexahydrate (12323, Alfa Aesar) became 15 mM, and then stirred for 7 min to obtain a nanowire growth solution. Here, two substrates on which a Cr oxide layer was deposited in the form of a flow path prepared by the above procedure were bonded to a 76 mm×52 mm×0.8 to 1.0 mm slide glass with a carbon tape, immersed in the growth solution, and heated in an air-blowing constant-temperature high-temperature apparatus at 95□ for 3 hours to grow nanowires. Subsequently, the substrate was removed from the beaker and washed away with ultrapure water to remove non-specifically grown nanowires.

(4) The substrate on which the nanowires manufactured in (3) above were grown was stuck on the petri dish. PDMS prepolymer (Silpot 184, Dow Corning Toray Ind., Ltd.) and the curing agent (Silpot 184 CAT,Dow Corning Toray Ind., Ltd.) were poured into the dish at a weight ratio of 10:1 and then mixed under conditions of 2000 rpm, 2 min, 2200 rpm, and 6 min. This was evacuated for 2 h to remove bubbles in the polymer, and then the polymerization proceeded by heating on a hot plate at 80□ for 2 h to cure the polymer. These operations embedded the nanowires on the Si-substrate into PDMS. PDMS in which these nanowires were embedded was exfoliated from the Si substrate, and PDMS embedded nanowires were stuck on the slide glass. Then, under the same condition as in (3) above, the nanowires embedded in PDMS were grown. After the growth, the embedded nanowires were removed from the beaker, and the non-specifically grown nanowires were removed by washing away with ultrapure water, thereby completing the manufacture of PDMS embedded nanowires.

(5) A negative-type photoresist (SU-8 3025, Nippon Kayaku Co., Ltd.) was applied on a silicon substrate by a spin-coater, covered with a photomask having a shape in which the flow path portion can be exposed, and exposed and developed to manufacture a mold in which the portion forming the flow path becomes convex. Next, the manufactured mold was placed in a petri dish. Next, a PDMS prepolymer and a curing agent similar to those described in (4) above were put in a container at a weight ratio of 10:1, and then mixed at the condition of 2000 rpm for 2 min, and 2200 rpm for 6 min, and it was poured into a petri dish and vacuum-drawn for 2 h to remove bubbles in the polymer. After 2 hours, the polymerization was proceeded by heating on a hot plate at 80□ for 2 hours to cure the polymer. The cured polymer was cut out, an introduction hole and a collection hole were opened with a punch of 0.32 mm in the flow path, to prepare a cover member. (6) Finally, on the substrate having nanowires manufactured in (4) above, the cover member manufactured in (5) above was placed. Further, a device 5 was manufactured by inserting PEEK tubes into the introduction hole and the collection hole and fixing the tubes with adhesive. FIG. 6E shows an enlarged photograph of the nanowires of the manufactured device 5.

### Extract and Analyze miRNA

### Example 1

Saliva was used as a sample and devices 1 to 4 were used as the device, and miRNA were extracted from EVs contained in saliva and analyzed by the following steps.

(1) Preparation of Sample Solution

Saliva was collected from subjects. In Example 1, saliva is used as it is, but in order to remove impurities in saliva, saliva may be placed in a centrifuge tube if necessary, and impurities may be removed by centrifugation. Note that this centrifugation is just for removing impurities and different from the ultracentrifugation for fractionating and collecting EVs.

(2) Capture of EVs in Samples 10 μl of saliva sample was dropped to devices 1 to 4 and allowing them to stand for about 10 seconds, to capture EVs in the saliva sample in the device. Next, the device was picked with tweezers and immersed in PBS for about 10 seconds to clean RNase and the like.

(3) Extraction of miRNA

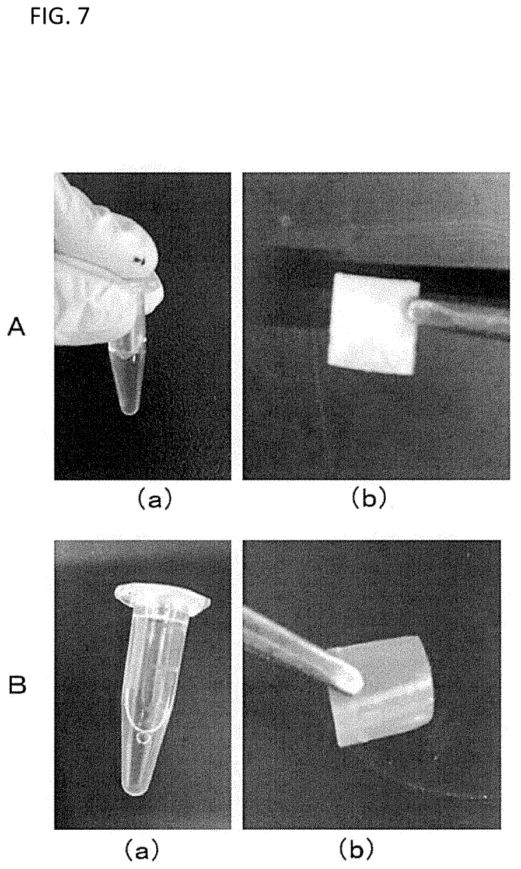

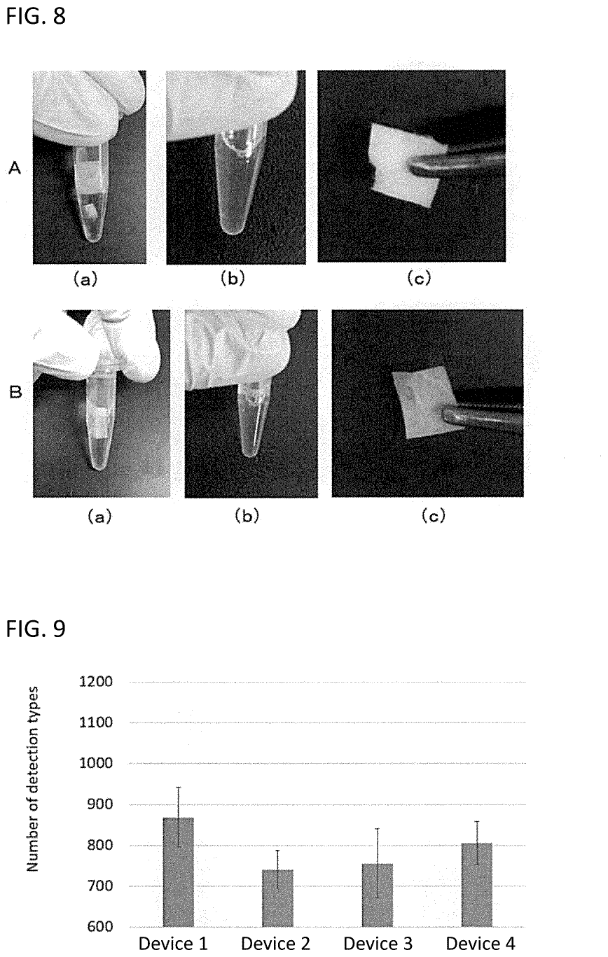

Cell lysis buffer M (038-21141, Wako) was used as the disruption solution. 1 ml of disruption solution was put in a centrifuge tube, and then the device in which EVs were captured in (2) above was introduced into a centrifuge tube, and after stirring for about 3 seconds by vortex, the device was allowed to stand for 5 minutes, whereby EVs were directly dissolved from the device in which EVs were captured, and the extraction of miRNA was performed. FIG. 7A shows a photograph when the device 1 was used: (a) a photograph of the centrifuge tube after removing the device 1 from the centrifuge tube after miRNA extraction; and (b) a photograph of the device 1 removed from the centrifuge tube. FIG. 7B shows a photograph when the device 2 was used: (a) a photograph of the centrifugal tube after removing the device 2 from the centrifugal tube after miRNA extraction; and (b) a photograph of the device 2 removed from the centrifugal tube. FIG. 8A shows a photograph when the device 3 was used; (a) a photograph of the centrifuge tube immediately after completion of miRNA extraction step; (b) a photograph of the centrifuge tube after removal of the device 3 from the centrifuge tube after miRNA extraction; and (c) a photograph of the device 3 removed from the centrifuge tube. FIG. 8B shows a photograph when device 4 was used: (a) a photograph of a centrifuge tube immediately after completion of miRNA extraction step; (b) a photograph of the centrifuge tube after removal of the device 4 from the centrifuge tube after miRNA extraction; and (c) a photograph of the device 4 removed from the centrifuge tube. As shown in FIG. 7A and FIG. 7B, when the device 1 and the device 2 made of cellulose nanofibers were used, no fibers or the like derived from the device were found in the centrifuge tube even after the EVs were disrupted by the disruption solution, and the removed device remained in its original shape. Therefore, after the extraction of miRNA, the miRNA extract could be produced simply by removing the device with tweezers.

On the other hand, as shown in FIG. 8A and FIG. 8B, when the device 3 and the device 4 made of pulp (cellulose fiber) were used, fibers separated from the device were seen in the centrifuge tube, and the removed device was partially defective. Therefore, when the device 3 and the device 4 were used, fibers derived from the device which became an obstacle as impurities at the time of miRNA analysis described later were removed by centrifugation.

(4) miRNA Analysis

Next, the types of miRNA contained in miRNA extract were analyzed using a 3D-Gene (registered trademark) (manufactured by Toray Industries, Ltd.) human miRNA chip by the following procedure.

(a) miRNA extract was purified using a SeraMi Exosome RNA purification column kit (System Biosciences Inc.) according to the kit manufacturer's instructions.

(b) 15 μl of purified miRNA extract was analyzed using a microarray and a 3D-Gene Human miRNA Oligo chip ver.21 (Toray Industries) for miRNA profiling. 3D-Gene contains 2565 human miRNA probes and can analyze expressions of up to 2565 miRNA types from miRNA extracts.

(c) The expression level of each miRNA in the miRNA extract was analyzed by calculating the background-subtracted signal intensity of all miRNA in each microarray, followed by a global normalization.

FIG. 9 shows a graph showing the types of miRNA contained in the miRNA extract extracted using the devices 1 to 4. Note that for each device an average value of three analysis results is shown. As shown in FIG. 9, it has been confirmed that miRNA can be extracted directly from the EVs captured in the devices even when any of the devices 1 to 4 was used. Also, as shown in FIG. 7 and FIG. 8, the device 3 and the device 4 manufactured of pulp, a portion of the device was defective during the EVs disruption, and fibers separated from the device were seen in the centrifuge tube. Therefore, it became clear that the device 1 and the device 2 are preferred when the extraction of miRNA from the sample solution is followed by analysis of miRNA

Note that, in the method of separating EVs by ultracentrifugation of conventional saliva and analyzing miRNA, 27 types of EVs were obtained as described on page 10 in the above Non-Patent Literature 2. In addition, in the above-mentioned Non-Patent Literature 3, as described in FIG. 7, 93 types of miRNA could be analyzed. In addition, in the method described in Non-Patent Literature 3, it is described that 5 ml or even 15 ml of saliva is used, but collecting such a large amount of saliva has a very large burden on the subject (patient). On the other hand, when the devices 1 to 4 were used, more than 700 types of miRNA were successfully analyzed using only 10 μl of saliva. In other words, it means that the trace amounts of miRNA as content could also be analyzed.

From the above results, when the device 1 to the device 4 are used, it is possible to simplify the extraction operation procedure of miRNA from the EVs in the saliva sample as compared with the separation method of the EVs using a conventional ultracentrifugation or the like. In addition, since miRNA can be directly extracted from the device that captured the EVs (and EVs in the saliva can be captured by the device at a high rate), the loss during miRNA extracting operation is reduced, and a remarkable effect was confirmed that miRNA analysis can be performed with high accuracy. Therefore, the extraction methods of miRNA disclosed in this application re very useful as a sample preparation method in the analysis method of miRNA contained in a sample solution. In addition, miRNA could be analyzed with high accuracy from saliva, which, as a biological sample solution, is difficult to collect in an invasive manner for a large amount. Thus it is expected that the cancer diagnosis is also carried out by contacting the devices 1 to 4 to tongues at the time of a medical examination or the like.

### Example 2

Urine was used as the sample solution, and a device 5 was used as the device, and miRNA were extracted and analyzed from EVs contained in the urine by the following procedure.

(1) Sample Preparation

1 mL of commercially available urine (Proteogenex, Bioreclamationl VT, EW Biopharma) was dispensed into a 1.5 mL centrifuge tube, and this centrifuge tube was set in a cooled centrifuge, and the impurities were precipitated by centrifugation at 3000×g for 15 min at 4□. In the following, the supernatant portion excluding this impurity is described as a urine sample.

(2) Capture of EVs in Urine Sample

1 mL of the above urine sample was introduced into the device 5 under the condition of a flow rate of 50 μL/min by a syringe pump, and EVs in the urine sample were captured by the nanowires.

(3) Extraction of miRNA

As disruption solution, the same cell lysis buffer M as in Example 1 was used, and the capture EVs were dissolved by introducing 1 mL of the disruption solution into the device, to prepare a miRNA extract.

(4) miRNA Analysis

Analysis was performed in the same manner as in Example 1.

228 experiments were performed in the same manner, and 1144 types of miRNA could be detected in average, using the devices 5. From the above results, it was confirmed that miRNA could be extracted directly from the EVs captured at the nanowires.

In addition, according to the method of separating EVs by conventional ultracentrifugation and analyzing miRNA, three experiments were carried out by the same procedure. 171, 261, and 352 types of miRNA could be analyzed. In addition, in an experiment using ExoQuick (manufactured by Funacosi Corporation), which is a commercially available EVs concentration kit using a resin based EVs adsorption carrier, 337, 355, and 491 types of miRNA could be analyzed.

From the above results, it was also confirmed that the same remarkable effect as described in Example 1 was obtained even when a biological sample other than saliva and a device for capturing EVs other than a device made of wood fiber were used.

From the above results, it was also confirmed that the same remarkable effect as described in Example 1 was obtained even when a biological sample other than saliva and a device for capturing EVs other than a device made of wood fiber were used.

### INDUSTRIAL APPLICABILITY

The method for extracting miRNA and the method for analyzing miRNA disclosed in the present application can extract and analyze miRNA from a sample solution in a simple and high-precision manner. Therefore, it is useful for cell experiments, etc. in medical institutions, universities, companies, research institutions, etc.

### REFERENCE SIGNS LIST

1, 1a-1b . . . device, 2, 2a . . . substrate, 3 . . . nanowire, 4 . . . cover member, 5 . . . catalyst layer, 6 . . . resist, 41 . . . cover member base material, 42 . . . flow path, 43 . . . sample introduction hole, 44 . . . sample collection hole, 45 . . . surface opposite to second surface, 46 . . . non-planar region, 47 . . . second surface

1. A method of extracting miRNA from extracellular vesicles in a sample solution using a device capable of capturing extracellular vesicles, the method comprising:
an extracellular vesicle capture comprising bringing the sample solution into contact with the device, to capture an extracellular vesicle in the device; and
a miRNA extraction comprising bringing the device that captured the extracellular vesicle with a disruption solution of extracellular vesicles, to disrupt the extracellular vesicle and extract miRNA from the extracellular vesicle into the disruption solution.

2. The method of extracting miRNA, according to claim 1, comprising: a device cleaning comprising cleaning the device that captured the extracellular vesicle, between the extracellular vesicle capture and the miRNA extraction.

3. The method of extracting miRNA, according to claim 1, wherein the device is formed of a material which is resistant to a disruption solution.

4. The method of extracting miRNA, according to claim 1, wherein the device comprises a nonwoven fabric comprising cellulose fibers.

5. The method of extracting miRNA, according to claim 4, wherein the cellulose fiber is a cellulose nanofiber.

6. The method of extracting miRNA, according to claim 3, wherein the device comprises at least one selected from:
nanowires,
a structure made with cellulose fibers, and
a structure made with cellulose nanofibers.

7. The method of extracting miRNA, according to claim 6, wherein the device is a structure made with cellulose nanofibers.

8. The method of extracting miRNA, according to claim 1, wherein the sample solution is a non-invasive biological sample solution.

9. The method of extracting miRNA, according to claim 8, wherein the sample solution is saliva.

10. A method of analyzing miRNA contained in an extracellular vesicle in a sample solution, comprising an analysis comprising analyzing miRNA contained in the disruption solution extracted by the miRNA extraction method of miRNA according to claim 1.
